SEQUENCE LISTING (6 sequences)

SEQ ID NO: 1 - DNA, 1297 bp, Arabidopsis thaliana
tcactataaagccgatccatagataaacgaggaccggccagaaatcgcttcaccattcccaaatctctcttccattttctccacacaaatttctcttcaatctccgataatggaaggaaccggcgttgtggcggtgtacggtaacggtgcgataacggaggcgaagaaatctcccttctccgtgaaggtcggtttggctcagatgctccgtggtggtgttatcatggatgtcgtcaacgccgagcaagctcgtatcgccgaggaggctggtgcttgcgccgtcatggctttggagcgtgttcctgctgatatccgcgctcaaggaggcgtcgctcgtatgagcgatccacaaatgattaaagaaatcaaacaagccgttacgattccggtgatggctaaggctaggattggtcatttcgttgaagctcagatccttgaagcaattggaatcgattacatcgatgagagcgaggttttgactcttgctgatgaagatcatcacatcaacaagcataatttccggatcccgttcgtttgcggttgccggaatctcggcgaggctctgaggaggatccgtgaaggtgcggcgatgattaggaccaaaggtgaagctggaaccggtaacattattgaagctgtgaggcatgtgaggtctgttaatggtgacattagggttttgcgaaacatggatgatgatgaggttttcactttcgctaagaaattagccgctccgtacgatctcgtgatgcagactaagcagcttggtcgtcttcctgtagtccaattcgccgccggtggagtggctactccggctgatgcagctctcatgatgcagcttggatgtgatggtgtctttgttggttctggtatcttcaagagcggtgacccagctcgtcgtgcacgtgccattgttcaggctgtgactcattacagtgaccctgagatgcttgtggaggtgagctgtgggcttggagaagccatggttgggatcaatctcaacgatgagaaggttgagaggttcgctaatcgctccgagtgatcaaagaaataaaaggtaaaatatctcagacgaaatggtttcagaattttctcagaccattttgcagtaatctctttgaaaagaagaagatgatgatattgttggtagtttgtatcctttgtgttttccttataatctttgatagtcttttgttattgtaactcgtaatccctttgcaagaacaagtttgtcagttataataatgtactactctcttgatcgatcagttggttttgaatctgatatattcttcgatcc

SEQ ID NO: 2 - protein, 309 aa, Arabidopsis thaliana
MEGTGVVAVYGNGAITEAKKSPFSVKVGLAQMLRGGVIMDVVNAEQARIAEEAGACAVMALERVPADIRAQGGVARMSDPQMIKEIKQAVTIPVMAKARIGHFVEAQILEAIGIDYIDESEVLTLADEDHHINKHNFRIPFVCGCRNLGEALRRIREGAAMIRTKFEAGTGNIIEAVRHVRSVNGDIRVLRNMDDDEVFTFAKKLAAPYDLVMQTKQLGRLPVVQFAAGGVATPADAALMMQLGCDGVFVGSGIFKSGDPARRAAAIVQAVTHYSDPEMLVEVSCGLGEAMVGINLNDEKVERFANRSE

SEQ ID NO: 3 - DNA, 30 bp, Artificial Sequence
  Sense Primer
gaagatctatggaaggaaccggcgttgtgg

SEQ ID NO: 4 - DNA, 32 bp, Artificial Sequence
  Antisense Primer
cgaagcttttataactgacaaacttgttcttg

SEQ ID NO: 5 - DNA, 29 bp, Artificial Sequence
  Sense Primer
gaagatctcactcggagcgattagcgaac

SEQ ID NO: 6 - DNA, 30 bp, Artificial Sequence
  Antisense primer
gctctagatggaaggaaccggcgttgtggc

(19) United States
(12) Patent Application Publication
(10) Pub. No.: US 2009/0133153 A1
(43) Pub. Date: May 21, 2009

(54) Polypeptide participating in pyridoxine biosynthesis, polynucleotide encoding the polypetide and those uses

(75) Inventors: Dong-hee Lee, Busan (KR); Tae-Hoon Kim, Busan (KR); In-taek Hwang, Daejeon (KR); Kwang-yun Oho, Daejeon (KR)

Correspondence Address: INTELLECTUAL PROPERTY LAW GROUP LLP, 12 SOUTH FIRST STREET, SUITE 1205, SAN JOSE, CA, 95113, US

(73) Assignee: Genomine, Inc, Pohang, Kyungbuk (KR)

(21) Appl. No.: 10/590,034

(22) PCT Filed: Feb. 18, 2005
(86) PCT No.: PCT/KR2005/000453

§ 371 Date: Aug. 18, 2006

(30) Foreign Application Priority Data
Feb. 20, 2004 (KR) 10-2004-0011517

(51) Int. Cl. C12N 15/82; C07K 14/415; C12Q 1/68; C12N 15/29

(52) U.S. Cl. 800/278; 530/379; 536/23.6; 435/6

(57) ABSTRACT

The present invention discloses a polypeptide participating in pyridoxine biosynthesis, a polynucleotide coding the polypeptide and those uses. Particularly, this present invention discloses a polypeptide participating in pyridoxine biosynthesis, a polynucleotide coding the polypeptide, a method for inducing plant growth inhibition, a method for screening a compound inducing plant growth inhibition, and composition for inducing plant growth inhibition which comprises the compound obtained by the screening method.

### FIELD OF THE INVENTION

The present invention relates to a polypeptide participating in pyridoxine biosynthesis, a polynucleotide encoding the polypeptide and those uses.

### BACKGROUND OF THE INVENTION

Pyridoxine is a compound named as 2-methyl-3-hydroxy-4,5-di(hydroxymethyl)pyridine and is essential for the growth of plants and animals (Gregory, J. F. Ann. Rev. Nutr. 18: 277-296, 1998). Pyridoxine belongs to vitamin B6 group together with other pyridoxine derivatives such as pyridoxamine and pyridoxal. These compounds are converted to pyridoxal-5-phosphate in a living body. It is reported that pyridoxal-5-phosphate is a coenzyme not only to participate in the nitrogen metabolism of all organisms, but also to play an important role in various metabolic actions of living cell including the amino acid metabolism.

The biosynthetic pathway of pyridoxine exists in plants, but is missed in animals and human (Dolphin, et al., In Vitamin B6 Pyridoxal Phosphate, 1986). In animals and human, pyridoxine should be ingested by food from outer environment.

Meanwhile, this fact that the biosynthetic pathway of pyridoxine exists in plant without exception but doesn't exist in animal and human has some significant meaning. The reason is that this suggests that the plant growth can be selectively suppressed by blocking the pyridoxine biosynthesis without any other toxicity for animals and human.

For this reason, the researchers engaging in plant biotechnology have tried to isolate polypeptides (enzymes) or polynucleotides (genes) engaging in the pyridoxine biosynthesis in plant.

In this background, the present invention has been completed.

### SUMMARY OF THE INVENTION

The object of the present invention is to provide a polypeptide participating in pyridoxine biosynthesis.

Another object of the present invention is to provide a polynucleotide encoding the polypeptide.

Still another object of the present invention is to provide a method for inhibiting plant growth.

Still another object of the present invention is to provide a process for screening a growth inhibitor of plants.

Still another object of the present invention is to provide a composition for inhibiting plant growth comprising the growth inhibitor screened by the process.

Other objects or aspects of the present invention are set forth hereinafter.

### BRIEF DESCRIPTION OF THE DRAWINGS

FIG. 1 depicts the amino acid sequence of the polypeptide having the pyridoxine biosynthesis function, preferably the amino acid sequence set forth in SEQ ID NO. 2 in an alignment of its amino acid sequences (SEQ ID NO. 2) and the amino acid sequence of KOG1606 family proteins as a stationery phase-induced protein including SOR/SNX family by using Clustal W method.

FIG. 2 depicts the expression of the polynucleotide encoding a polypeptide having the pyridoxine biosynthesis function, in cell extracts of E. coli transformant transformed with the recombinant vector containing the nucleotide sequence of SEQ ID. NO. 1 and control group transformant by performing SDS-PAGE analysis.

FIG. 3a depicts the schematic construction of a cloning vector before the polynucleotide encoding a polypeptide having the pyridoxine biosynthesis function, preferably the nucleotide sequence of SEQ ID NO. 1 is inserted into the vector in an anti-sense direction.

FIG. 3b depicts the schematic construction of the recombinant vector into which the polynucleotide encoding a polypeptide having the pyridoxine biosynthesis function, preferably the polynucleotide of SEQ ID. NO. 1 is inserted in an anti-sense direction.

FIG. 4 depicts the plant saplings germinated from T1 seeds of Arabidopsis transformant into which the anti-sense nucleotide against a polynucleotide encoding a polypeptide having the pyridoxine biosynthesis function, preferably the polynucleotide of SEQ ID. NO. 1 is introduced (saplings that are cultivated and differentiated after treating a herbicide, Basta).

FIG. 5a depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes after adding pyridoxine).

FIG. 5b depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes without pyridoxine).

FIG. 5c depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 19 days (saplings that are cultivated and differentiated on petri dishes after adding pyridoxine).

FIG. 5d depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 19 days (saplings that are cultivated and differentiated on petri dishes without pyridoxine).

FIG. 6a depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes without any other vitamin B6's).

FIG. 6b depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes after adding pyridoxine).

FIG. 6c depicts the plant saplings germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes after adding pyridoxamine).

FIG. 6d depicts the plant saplings that are germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes after adding pyridoxal).

FIG. 6e depicts the plant saplings that are germinated from T2 seeds of Arabidopsis transformant into which the anti-sense nucleotide against a polynucleotide encoding a polypeptide having the pyridoxine biosynthesis function, preferably the polynucleotide of SEQ ID. NO. 1 is introduced, and cultivated for 7 days (saplings that are cultivated and differentiated on petri dishes after adding pyridoxal-5-phosphate).

### DETAILED DESCRIPTION OF THE INVENTION

### Technical Solution

In one aspect, the present invention provides a polypeptide participating in pyridoxine biosynthesis.

As demonstrated in the following Examples, the present inventors, using the primers manufactured based upon the amino acid sequence of a polypeptide deduced to participate in the pyridoxine biosynthesis in Arabidopsis thaliana (GeneBank accession number: NP 195761), constructed full-length cDNA of Arabidopsis thaliana, and determined the nucleotide sequence of the cDNA, that is, the nucleotide sequence of SEQ ID NO. 1, and estimated the molecular weight of the polypeptide encoded by the cDNA by deducing the amino acid sequence, that is, the amino acid sequence of SEQ ID NO. 2, based upon open reading frame of the cDNA, and induced the polypeptide to be expressed in E. coli. by introducing the recombinant vector containing the cDNA fragment. As a result, the present inventors became to know that the molecular weight of the expressed polypeptide is identical to the estimated molecular weight and that a pyridoxine auxotroph mutant is obtained by introducing the anti-sense nucleotide, manufactured on the basis of the cDNA sequence, that is, the nucleotide sequence of SEQ ID. NO. 1, into Arabidopsis thaliana. Further, the present inventors also became to know that the pyridoxine auxotroph mutant can recover its phenotype by treating pyridoxine among vitamin B6 group, excluding pyridoxine, pyridoxal, pyridoxal-5-phosphate or the like, and that the polypeptide participates directly in the pyridoxine biosynthesis.

Therefore, as used herein, “participating in pyridoxine biosynthesis” or “having the pyridoxine biosynthesis function” means “indispensable for pyridoxine biosynthesis.”

In detail, the polypeptide participating in pyridoxine biosynthesis according to the present invention is a member selected from a group consisting of (a), (b) and (c) polypeptide:
(a) polypeptide containing all portion of the amino acid sequence set forth in SEQ ID NO. 2;
(b) polypeptide containing a substantial portion of the amino acid sequence set forth in SEQ ID NO. 2;
(c) polypeptide substantially similar to the above (a) or (b) polypeptide

As used herein, a “polypeptide containing a substantial portion of the amino acid sequence set forth in SEQ ID. NO. 2” refers to a polypeptide containing the partial portion of amino acid sequence of SEQ ID NO. 2 that still retains the pyridoxine biosynthesis function, compared with a polypeptide consisting of the amino acid sequence of SEQ ID NO. 2. A polypeptide retaining the pyridoxine biosynthesis function is regarded as the polypeptide of the present invention, regardless of the polypeptide's length or activity degree. That is to say, any polypeptide retaining the pyridoxine biosynthesis function can be a “polypeptide containing a substantial portion of the amino acid sequence set forth in SEQ ID NO. 2” even if it is shorter in the length or lower in the enzymatic activity than the polypeptide consisting of the amino acid sequence of SEQ ID NO. 2. Even though any amino acid sequence is partially deleted from or added to the amino acid sequence of SEQ ID NO. 2, those skilled in the arts may expect that the deleted or added polypeptide can still retains the pyridoxine biosynthesis function. For example, the polypeptides that are deleted in the N-terminus and/or the C-terminus belong to such a polypeptide. It is already well known in prior arts that polypeptides can often retain the intrinsic activity, even though deleted in the N-terminus and/or the C-terminus. Depending upon cases, the polypeptide deleted in the N-terminus and/or the C-terminus may not retain the enzymatic activity, since the termini are an essential motif of enzyme, but those skilled in the art can discriminate or detect the active polypeptide from inactive polypeptides within the ordinary knowledge. Also, even if any other region as well as the termini is deleted, the deleted polypeptides can still retain the intrinsic activity. Those skilled in this art can decide whether the deleted polypeptides still retain the intrinsic activity or not, within the ordinary knowledge. Particularly, the nucleotide sequence of SEQ ID NO. 1 and the amino acid sequence of SEQ ID NO. 2 are disclosed in the specification. The polypeptide that is encoded by the nucleotide sequence of SEQ ID NO. 1 and consists of the amino acid sequence of SEQ ID NO. 2 is clarified to have the pyridoxine biosynthesis function in the following Examples. Therefore, it is obvious that those skilled in the art can identify easily whether or not the polypeptide partially deleted in the amino acid sequence of SEQ ID NO. 2 still retain the intrinsic activity of the polypeptide consisting of the amino acid sequence of SEQ ID NO. 2, within the ordinary knowledge. Hence, it is naturally understood that a “polypeptide containing a substantial portion of the amino acid sequence set forth in SEQ ID NO. 2” includes all deleted polypeptides that still retain the pyridoxine biosynthesis function and can be easily manufactured by those skilled in the art within the ordinary knowledge on a basis disclosed in the specification.

As used herein, a “polypeptide substantially similar to above (a) or (b) polypeptide” refers to a polypeptide that still retains the function of polypeptide consisting of the amino acid sequence of SEQ ID NO. 2, that is, the pyridoxine biosynthesis function, even if one or more amino acids are substituted. At this time, insofar as any polypeptide still retains the pyridoxine biosynthesis function, the polypeptide's activity or substitution degree is of little importance. That is to say, any substituted polypeptide still retaining the pyridoxine biosynthesis function can be within the scope of the present invention, even though the polypeptide has many substituted amino acids or is much lower activity than that of the polypeptide consisting of the amino acid sequence of SEQ ID NO. 2. In reference, even if one more amino acids are substituted, the polypeptide substituted in one more amino acids may retain the intrinsic activity of a polypeptide if the substituting amino acids are chemically equivalent to the substituted amino acids. If a hydrophobic amino acid such as alanine is displaced with other hydrophobic amino acids such as glycin or more hydrophobic amino acids such as valine, leucine or isoleucine, the polypeptide substituted in one more amino acids may retain the intrinsic function of a polypeptide, in spite of activity reduction. In addition, if a negative-charged amino acid such as glutamic acid is displaced with other negative-charged amino acids such as aspartic acid, the polypeptide substituted in one more amino acids may retain the intrinsic function of a polypeptide. Besides, if a positive-charged amino acid such as arginine is displaced with other positive-charged amino acids such as lysine, the polypeptide substituted in one more amino acids may retain the intrinsic function of a polypeptide, in spite of activity reduction. Furthermore, even if the N-terminus or the C-terminus is substituted, the polypeptide can retain the intrinsic activity. Those skilled in this art can easily manufacture the polypeptide that still retains the function of the polypeptide consisting of the amino acid sequence of SEQ ID NO. 2, even if substituted in one more amino acids, and decide whether or not this polypeptide still retains the intrinsic activity. Particularly, the nucleotide sequence of SEQ ID NO. 1 and the amino acid sequence of SEQ ID NO. 2 are disclosed in the specification, and the polypeptide consisting of the amino acid sequence of SEQ ID NO. 2 is confirmed to have the pyridoxine biosynthesis function in the following Examples. Therefore, it is comprehended that those skilled in the art can accomplish a “polypeptide substantially similar to above (a) or (b) polypeptide” easily. Also in the present invention, a “polypeptide substantially similar to above (a) or (b) polypeptide” includes all polypeptides that is substituted in one more amino acids but retains the pyridoxine biosynthesis function.

As described in the above, a “polypeptide substantially similar to above (a) or (b) polypeptide” means all polypeptides that are substituted in one more amino acids and still retains the pyridoxine biosynthesis function. However, judging from the enzymatic activity, the polypeptide is preferable to become higher in the sequence homology, compared with the amino acid sequence of SEQ ID NO. 2. Preferably, the polypeptide has over 60% of sequence homology in the minimum while it has exactly 100% of sequence homology in the maximum.

In detail, the above sequence homology is preferable to become higher in the order of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

Because a “polypeptide substantially similar to above (a) or (b) polypeptide” includes not only a “polypeptide substantially similar to the polypeptide containing all portion of the amino acid sequence set forth in SEQ ID NO. 2” but also a “polypeptide substantially similar to the polypeptide containing a substantial portion of the amino acid sequence set forth in SEQ ID NO. 2” in the present invention, all the above-described description applies to a “polypeptide substantially similar to the polypeptide containing all portion of the amino acid sequence set forth in SEQ ID NO. 2” but also a “polypeptide substantially similar to the polypeptide containing a substantial portion of the. amino acid sequence set forth in SEQ ID NO. 2.”

In another aspect, the present invention provides an isolated polynucleotide encoding above-mentioned polypeptide. At this moment, an “above-mentioned polypeptide” has the meaning to include the polypeptide that has the pyridoxine biosynthesis function and contains all portion of the amino acid sequence set forth in SEQ ID NO. 2, the polypeptide that has the pyridoxine biosynthesis function and contains a substantial portion of the amino acid sequence set forth in SEQ ID NO. 2, and the polypeptide that has the pyridoxine biosynthesis function and is substantially similar to any one of above-mentioned polypeptide. The “above-mentioned polypeptide” also includes all preferable polypeptides described above. Therefore, the polynucleotide of the present invention includes an isolated polynucleotide encoding the polypeptide that has the pyridoxine biosynthesis function and contains all portion of the amino acid sequence set forth in SEQ ID NO. 2, an isolated polynucleotide encoding the polypeptide that has the pyridoxine biosynthesis function and contains a substantial portion of the amino acid sequence set forth in SEQ ID NO. 2, and an isolated polynucleotide encoding the polypeptide that has the pyridoxine biosynthesis function and is substantially similar to any one of above-mentioned polypeptide. The polynucleotide of the present invention further includes the isolated polynucleotide encoding all the polypeptides that have the pyridoxine biosynthesis function and have the sequence homology in the above-illustrated order. It is natural that those skilled in the art can manufacture conveniently the polynucleotide encoding the amino acid sequence on a basis of its sequence if available.

On the other hand, an “isolated polynucleotide” defines to include a polynucleotide synthesized chemically, a polynucleotide separated from organism such as Arabidopsis thaliana and a polynucleotide containing modified nucleotides, and also include single-stranded or double-stranded RNA or DNA polymers. Particularly, an “isolated polynucleotide” includes genomic DNA separated from Arabidopsis thaliana as well as cDNA and chemically synthesized polynucleotide. It is natural within the common knowledge that those skilled in the art can synthesize a polynucleotide chemically, manufacture cDNA, purify genomic DNA and the like, on a basis of prior arts, the amino acid sequence of SEQ ID NO. 2, and the nucleotide sequence of SEQ ID NO. 1 encoding the same as disclosed in the specification.

In another aspect, the present invention provides a polynucleotide containing the nucleotide sequence of SEQ ID NO. 1 partially or a polynucleotide substantially similar to the polynucleotide. At this moment, a “polynucleotide containing the nucleotide sequence of SEQ ID NO. 1 partially” defines a polynucleotide containing a partial nucleotide sequence sufficient to identify and/or purify a gene having the pyridoxine biosynthesis function from organism such as Arabidopsis thaliana and other plants. Besides, “polynucleotide substantially similar to the polynucleotide” defines a polynucleotide that is substituted in one or more nucleotides, compared with the partial region of nucleotide sequence of SEQ ID NO. 1, but maintains the sequence-dependent binding specificity sufficient to identify and/or purify a gene having the pyridoxine biosynthesis function from organism such as Arabidopsis thaliana and other plants.

It is natural within the common knowledge that those skilled in the art can identify and/or purify a gene having the pyridoxine biosynthesis function from organism such as Arabidopsis thaliana and other plants on a basis of prior arts and the nucleotide sequence of SEQ ID NO. 1 as disclosed in the specification.

Therefore, the polynucleotide of the present invention can include any polynucleotide that carries the sequence length or the binding specificity sufficient to identify and/or purify a gene having the pyridoxine biosynthesis function from organism such as Arabidopsis thaliana and other plants, regardless of length or homology to the nucleotide sequence of SEQ ID NO. 1.

Generally, it is reported that the nucleotide sequence contiguous over 30 nucleotides is required in order to putatively identify an unknown gene in comparison with the known gene and to be utilized as a probe for the purification of unknown gene. Therefore, the polynucleotide of the present invention is preferable to include more than 30 nucleotides adopted from the nucleotide sequence of SEQ ID NO. 1. However, the polynucleotide (or oligonucleotide) containing less than 30 nucleotides can also be within the scope of the present invention. The reason is that it may be sufficient to identify and/or purify a gene having the pyridoxine biosynthesis function from organism such as Arabidopsis thaliana and other plants, if it has 100% sequence homology to the corresponding portion of the nucleotide sequence of SEQ ID NO. 1 or is used in identification and/or purification under stringent condition (adjusting pH or concentration of buffer solution).

It is natural within the common knowledge that those skilled in the art can manufacture and detect the polynucleotide containing less than 30 nucleotides sufficient to identify and/or purify a gene having the pyridoxine biosynthesis function from organism such as Arabidopsis thaliana and other plants, and, using the polynucleotide, identify and/or purify a gene having the pyridoxine biosynthesis function from organism such as Arabidopsis thaliana and other organisms.

In another aspect, the present invention provides an anti-sense nucleotide that can bind complementarily to above-mentioned polynucleotide.

The anti-sense nucleotide of the present invention includes all poly (or oligo) nucleotides that can bind complementarily against a polynucleotide so as to inhibit the transcription (in DNA) or the translation (in RNA). If the anti-sense nucleotide binds complementarily against a polynucleotide encoding a polypeptide having the pyridoxine biosynthesis function to inhibit the transcription (in DNA) or the translation (in RNA), its length and complementary sequence homology is of little matter. Even a small polynucleotide comprising less than 30 nucleotides may be used as an anti-sense nucleotide, if it has 100% complementary sequence homology against its target sequence and is used under a proper condition by adjusting concentration, pH or the like. Even a complementary sequence under 100% complementary sequence homology may also become an anti-sense nucleotide, if it has a proper length. Therefore, it is natural that the anti-sense nucleotides of the present invention includes all anti-sense nucleotides that can retain the anti-sense activity to inhibit the transcription or the translation, regardless of length and complementary sequence homology degree.

It is natural within the common knowledge that those skilled in the art can determine the proper length and complementary sequence homology of an anti-sense nucleotide, and manufacture the anti-sense nucleotide on a basis of prior arts, the nucleotide sequence of SEQ ID NO. 1 and the amino acid sequence of SEQ ID NO. 2 as disclosed in the specification.

Preferably, the anti-sense nucleotide of the present invention can be an anti-sense nucleotide containing the partially complementary region against the nucleotide sequence of SEQ ID NO. 1. It is understood in the present invention that an “anti-sense nucleotide containing partially complementary region against the nucleotide sequence of SEQ ID NO. 1” means an anti-sense nucleotide that is sufficiently long to bind complementarily against DNA consisting of the nucleotide sequence of SEQ ID NO. 1 or RNA transcribed from the same so as to inhibit the transcription or the translation, as illustrated above.

In another aspect, the present invention provides a recombinant vector containing above-mentioned polynucleotide and a transformant transformed with the recombinant vector.

As described in following Examples, the polynucleotide that consists of the nucleotide sequence of SEQ ID NO. 1 and encodes the polypeptide having the pyridoxine biosynthesis function is introduced to the cloning vector pCAL-n (Stratagene, USA) to construct the recombinant vector pCAtPDX4.

After that, the recombinant vector pCAtPDX4 is transformed to E. coli and the resulting transformant is cultivated and induced to express a polypeptide in order to determine the molecular weight of the polypeptide. As a result, the polypeptide is identified to be the same in the molecular weight with the polypeptide estimated from the open reading frame of SEQ ID NO. 1.

In a preferred embodiment, the present invention provides the recombinant vector pCAtPDX4 and the E. coli transformant that is transformed with the recombinant vector.

In another aspect, the present invention provides a method for inhibiting plant growth. In detail, the method for inhibiting plant growth comprises step of inhibiting the expression or function of a polypeptide that has the pyridoxine biosynthesis function and consists of the amino acid sequence of SEQ ID NO. 2 or its equivalent sequence.

As used herein, “inhibiting plant growth” defines “causing retard of plant growth or plant lethality”

As demonstrated above, the biosynthetic pathway of pyridoxine exists in plants, but is missed in animals, even though pyridoxine is essential for the growth of plants and animals. Therefore, if the polypeptide having the pyridoxine biosynthesis function is not expressed and functionally suppressed, the plant growth may be inhibited. As disclosed in following Examples, Arabidopsis thaliana is transformed by using an anti-sense nucleotide complementary to the nucleotide sequence of SEQ ID NO. 1 and observed to retard in the growth, to seriously cause yellows in whole leaves, to become higher in the lethality or the like. Therefore, the method for inhibiting plant growth of the present invention adopts the step of inhibiting the expression of a polypeptide having the pyridoxine biosynthesis function or suppressing the function thereof.

As used herein, a “polypeptide consisting of the amino acid sequence of SEQ ID NO. 2 or its equivalent sequence” means to include all polypeptides that are derivatives to the polypeptide consisting of the amino acid sequence of SEQ ID NO. 2, and retain the pyridoxine biosynthesis function and evolve according to plant sorts to contain the amino acid sequence varied from the amino acid sequence of SEQ ID NO. 2. Therefore, it is naturally understood in the present invention that the scope of plant in the method for inhibiting plant growth may include not only Arabidopsis thaliana but also any other plants, even though the polypeptide consisting of the amino acid sequence of SEQ ID NO. 2 is isolated form Arabidopsis thaliana. The polypeptide consisting of the amino acid sequence of SEQ ID NO. 2 or its equivalent sequence is preferable to become higher in the sequence homology, naturally most preferable to have 100% of sequence homology, compared with the amino acid sequence of SEQ ID NO. 2. However, the polypeptide is preferable to have at least 60% of sequence homology in the minimum.

In detail, the above sequence homology is preferable to become higher in the order of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In the meantime, it is natural that those skilled in the art can exploit prior arts to inhibit the expression of polypeptide, preferably by using insertion of anti-sense nucleotide, gene deletion, gene insertion, T-DNA insertion, homologous recombination or transposon tagging, small interfering RNA (siRNA) and the like.

As described in the following Examples, in order to inhibit the expression of polypeptide, the anti-sense nucleotide is introduced to a plant by the process comprising steps: (1) manufacturing an anti-sense nucleotide against the polynucleotide of SEQ ID NO. 1; (2) constructing the recombinant vector containing the same; (3) transforming the recombinant vector to Agrobacterium tumefaciens; and (4) transforming the resulting transformant to Arabidopsis thaliana. As the result of cultivating a seed of Arabidopsis thaliana transformant, it is confirmed that the transformant retards in the growth remarkably, causes seriously yellows in whole leaves, becomes higher in the lethality or the like (See Example 3).

Therefore, in the method for inhibiting plant growth, preferably an anti-sense nucleotide containing a partially complementary region against the nucleotide sequence of SEQ ID NO. 1 can be introduced to a target plant in order to inhibit the expression of polypeptide. More preferably, a transformant transformed with the recombinant vector containing an anti-sense nucleotide against the nucleotide sequence of SEQ ID NO. 1 can be introduced into a target plant. Most preferably, the transformant can be Agrobacterium tumefaciens transformed with the recombinant vector containing the same. At this moment, an “anti-sense nucleotide containing partially complementary region against the nucleotide sequence of SEQ ID NO. 1” is the same as already described above in connection with the anti-sense nucleotide of the present invention.

Generally, it is reported that an anti-sense nucleotide may bind onto a target nucleotide array within a nucleotide sequence (RNA or DNA) to inhibit the function of nucleic acids or the expression. That is to say, the anti-sense nucleotide against specific gene sequence can hybridize both RNA and DNA so as to inhibit the expression of specific gene in transcription or translation.

Therefore, if the polypeptide having the pyridoxine biosynthesis function does not work properly by inhibiting the expression or function of the polypeptide consisting of the amino acid sequence of SEQ ID NO. 2 or its equivalent sequence, that can result in inhibiting the plant growth.

The method for inhibiting plant growth of the present invention is harmless to human or animals, since it exploits the biosynthetic pathway of pyridoxine that exists in plants but is missed in human or animals, to inhibit the production of pyridoxine.

In another aspect, the present invention provides a process for screening a growth inhibitor of plants, which comprises the step of screening a substance inhibiting the expression or function of a polypeptide having the pyridoxine biosynthesis function and consisting of the amino acid sequence of SEQ ID NO. 2 or its equivalent sequence.

As used herein, a “polypeptide having the pyridoxine biosynthesis function and consisting of the amino acid sequence of SEQ ID NO. 2 or its equivalent sequence” is the same as already explained in connection with the method for inhibiting plant growth according to the present invention.

Preferably, the growth inhibitor of the present invention can be an anti-sense nucleotide containing a partially complementary region against the nucleotide sequence of SEQ ID NO. 1; more preferably, a transformant transformed with the recombinant expression vector comprising the anti-sense nucleotide containing a partially complementary region against the nucleotide sequence of SEQ ID NO. 1; and most preferably, Agrobacterium tumefaciens transformant transformed with the recombinant expression vector. At this moment, an “anti-sense nucleotide containing partially complementary region against the nucleotide sequence of SEQ ID NO. 1” is the same as already described above in connection with the anti-sense nucleotide of the present invention.

In another aspect, the present invention provides a composition for inhibiting plant growth comprising the growth inhibitor screened by the above-described screening process.

Particularly, the growth inhibitor can be selected from a group comprising (1) an anti-sense nucleotide complementary to the polynucleotide of SEQ ID NO. 1; (2) a recombinant vector containing the anti-sense nucleotide; and (3) a transformant transformed with the recombinant vector, and preferably, Agrobacterium tumefaciens transformed with the recombinant vector.

### Peculiar Effect

According to the present invention, the polypeptide participating in pyridoxine biosynthesis, the polynucleotide encoding the polypeptide, the method for inhibiting plant growth, the process for screening a growth inhibitor of plants, and the composition for inhibiting plant growth comprising the growth inhibitor screened by the process are provided.

Hereinafter, the present invention will be described in reference to preferred embodiments. However, the scope of the present invention is not limited to these embodiments.

### EXAMPLE 1

### Separation of Polynucleotide Encoding Polypeptide Functionally Related to Pyridoxine Biosynthesis from Arabidopsis thaliana

In order to separate a gene encoding a polypeptide having the pyridoxine biosynthesis function, the screening is performed by using Arabidopsis thaliana as explained below.

<1-1> Cultivation and Culture of Arabidopsis thaliana

Arabidopsis thaliana was cultivated in a soil-filled pot or cultivated in a petri dish by using MS medium (Murashige and Skoog salts, Sigma, U.S.A.) containing 2% sucrose (pH 5.7) and 0.8% agar. In this study, vitamin B6 group (including pyridoxine) was excluded from the vitamin mixture used in the MS medium. The culture pot was incubated at 22° C. in a growth chamber and controlled by 16/8 hours of light and dark cycle.

<1-2> RNA Purification and Construction of cDNA Library

In order to construct cDNA library, Arabidopsis thaliana leaves were collected in various stages of differentiation to separate total RNAs by using TRI reagent (Sigma, USA). Then, total RNAs were purified to obtain poly(A)+ RNA according to the protocol of mRNA purification kit (Pharmacia, USA). The poly(A)+ RNA was utilized to prepare the double stranded cDNA by Not I-(dT)₁₈ as primer through cDNA synthesis kit (Time Saver, Pharmacia, USA).

<1-3> Purification of Gene Encoding the Polypeptide Having the Pyridoxine Biosynthesis Function

In order to separate gene encoding the polypeptide pyridoxine biosynthesis function, the primer of SEQ ID NO. 3 containing the recognition site of restriction enzyme Bgl II and the reverse primer of SEQ ID NO. 4 containing the recognition site of restriction enzyme Hind III were synthesized, based upon the amino acid sequence of the polypeptide that is deduced to relate to the pyridoxine biosynthesis in Arabidopsis thaliana (GeneBank accession number NP 195761). By performing PCR using the resulting primers, the full-length cDNA was amplified and isolated from the cDNA library of Arabidopsis thaliana constructed in Example 1-2.

As the result of analyzing the isolated cDNA, it is identified that the cDNA sequence contains the open reading frame (ORF) in 930 bps size, encodes 309 amino acids totally having 33.2 kDa molecular weight and is composed of one exon. The gene of the present invention has been named as AtPDX4 (Arabidopsis thaliana pyridoxine biosynthesis protein 4).

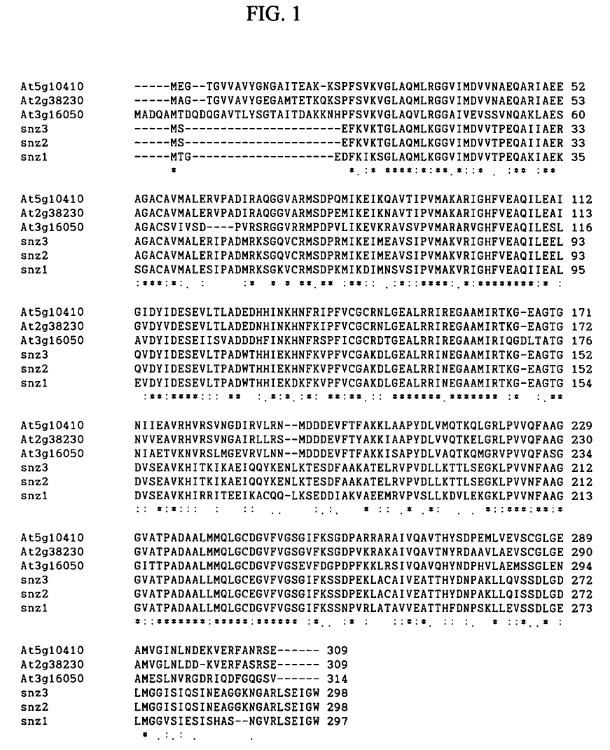

The amino acid sequence deduced from the AtPDX4 gene is determined to contain SOR/SNZ family domain in NO. 18˜226 of amino acid region. Thus, the polynucleotide of the present invention is concluded to participate in the pyridoxine biosynthesis and to regulate the defense mechanism against active oxygen. Therefore, this gene is guessed to protect a plant from various stresses such as activated oxygen or the like by the over-expression. Recently, it is also reported about this motif that most of amino acid sequences in this gene contains KOG 1606 family that is an arrest induction protein containing SOR/SNZ family. As illustrated in FIG. 1, the amino acid sequences of proteins belonging to this family were analyzed to compare with the amino acid sequence deduced from the AtPDX4 gene by the multiple sequences mode. In FIG. 1, At5g10410 denotes AtPDX4 protein of Arabidopsis thaliana; At3g16050, protein relating to ethylene induction protein (GeneBank accession number NP 188226); At2g28230 denotes protein relating to SOR 1 of Cercospora nicotiane in Arabidopsis thaliana (GeneBank accession number NP 18158); and SNZ 1, SNZ 2 and SNZ 3 denotes SNZ 1, SNZ 2 and SNZ 3 proteins of Saccharomyces cerevisiae (respectively, GeneBank accession number Q03143, P53824, P43545). Besides, the amino acid sequence is indicated by “*” in the same region; “:” in the conserved substitution region; and “.” in the semi-conserved substitution region. Furthermore, above-mentioned proteins were compared to measure the sequence homology of amino acids. As a result, the amino acid sequence of AtPDX4 gene had the sequence homology to 62% and 89% independently with the protein relating to ethylene induction protein and the protein relating to SOR 1 of Cercospora nicotiane in Arabidopsis thaliana; and sequence homology to 58%, 61% and 61% respectively with SNZ 1, SNZ 2 and SNZ 3 proteins of Saccharomyces cerevisiae. According to the motif analysis, it is elucidated that AtPDX4 gene of the present invention has the higher homology with both Pdx 1 and Pdx 2 gene. But, it is unclear which is more similar to AtPDX4 gene functionally and will be studied deeply in the future.

### EXAMPLE 2

### Purification of Protein Expressed by AtPDX4 Gene in E. coli

The DNA fragment containing full-length cDNA region of AtPDX4 gene that was amplified and isolated in Example 1-3 was digested by using the restriction enzymes BglII and HindIII and inserted to the recognition site of restriction enzymes BamHI (compatible end of BglII) and HindIII in the cloning vector pCAL-n (Stratagene, USA) to construct the recombinant vector pCAtPDX4. At this moment, the cloning vector pCAL-n is advantageous to contain the tag sequence of calmodulin-binding peptide and the exogenous protein expressed can be separated easily by using a calmodulin resin.

After that, the recombinant vector pCAtPDX4 was transformed to E. coli BL21-Gold(DE3) (Stratagene, USA), cultivated at 37° C. with agitation at 150 rpm in LB broth (Luria-Bertani broth, USB, USA) containing 100 g/ml of ampicillin until O.D. 600 value reached 0.7. In order to induce the intracellular expression of target protein in E. coli, isopropyl-D-thiogalactoside (IPTG) was added to the cell suspension to adjust final concentration to 1 mM and cultivated for 2 hours. The resulting cell was washed by using 50 mM potassium phosphate buffer (pH 7.0) containing 50 mM of MgSO₄ and 0.4 M of NaCl, and centrifuged at 4,000×g for 15 minutes, and then the precipitate was collected to be stored at −20° C.

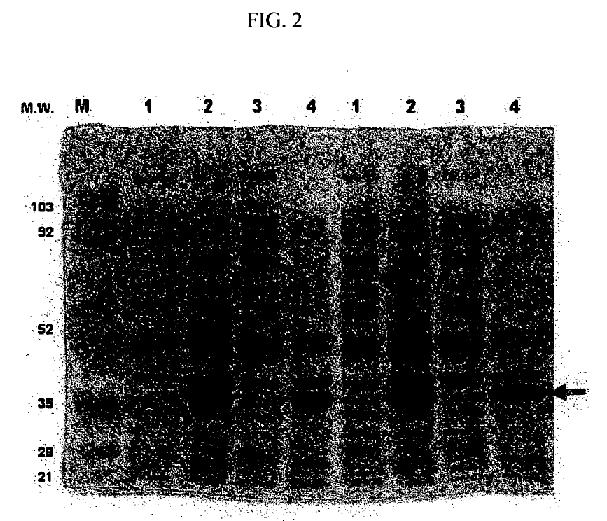

In order to identify the expression of the exogenous protein, the cell extract obtained from E. coli transformant transformed with the recombinant vector pCAtPDX4 was examined by electrophoresis on SDS-polyacrylamide gels (SDS-PAGE). As a result, it was observed to contain the fusion protein in about 37 kDa (33.2 kDa molecular weight of the protein expressed from AtPDX4 gene+4 kDa of calmodulin biding peptide) as depicted in FIG. 2. To the contrary, E. coli extract of control group was detected not to contain the protein in this size. In FIG. 2, the fusion protein in about 37 kDa size (33.2 kDa molecular weight of the protein expressed from AtPDX4 gene+4 kDa of calmodulin biding peptide) is indicated by arrow (.). Lanes 1 and 3 are E. coli extract of control group; lane 2, extract of E. coli colony-1 transformed with the recombinant vector containing AtPDX4 gene; and lane 4, extract of E. coli colony-2 transformed with the recombinant vector containing AtPDX4 gene.

### EXAMPLE 3

### Preparation and Characterization of Arabidopsis thaliana Transformant Inserted with Anti-Sense Construct Against AtPDX4 Gene

<3-1> Preparation of Arabidopsis Transformant Inserted with Anti-Sense Construct Against AtPDX4 Gene

In order to examine the physiological characteristics of protein isolated in the above Example 2, Arabidopsis thaliana transformant into which the AtPDX4 gene is inserted in the anti-sense direction was prepared to suppress the expression of AtPDX4 transcript.

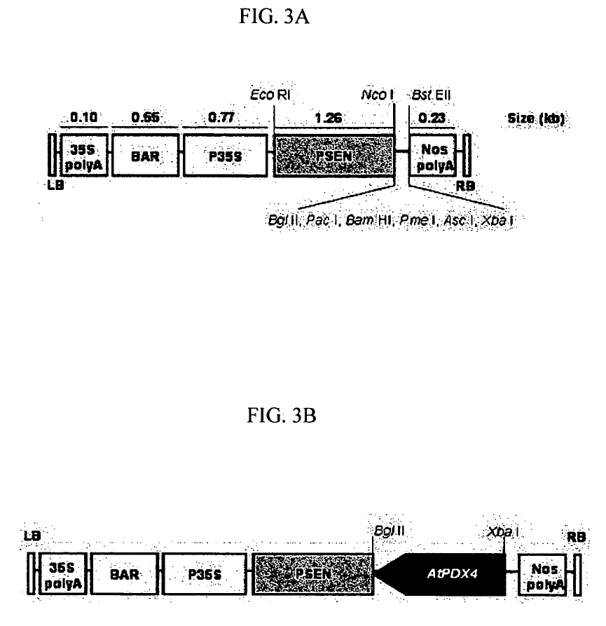

AtPDX4 cDNA was amplified in the anti-sense direction through PCR from Arabidopsis thaliana cDNA, by using a forward primer of SEQ ID NO. 5 containing the recognition site of restriction enzyme BglII and reverse primer of SEQ ID NO. 6 containing the recognition site of restriction enzyme XbaI. The resulting DNA fragment was digested by restriction enzyme BglII and XbaI, ligated to the cloning vector pSEN controlled by the promoter of sen 1 that is a stress- or aging-related gene to prevent plant death during the germination, and the recombinant vector pSEN-AtPDX4 as an anti-sense construct against AtPDX4 gene was manufactured. sen 1 promoter is specific for a plant gene expressed according to growth stages. In FIG. 3, the construction of the cloning vector pSEN (See FIG. 3a) and the recombinant vector pSEN-AtPDX4 (See FIG. 3b) were illustrated. As depicted in FIG. 3, BAR indicates the bar gene conferring resistance to herbicide BARSTA (phosphinothricin acetyltransferase gene); RB, right border; LB, left border; P35S, CaMV 35S RNA promoter; 35S poly A, CaMV 35S RNA poly A; PSEN, sen 1 promoter; Nos poly A, poly A of nopaline synthase gene.

The recombinant vector pSEN-AtPDX4 was introduced into Agrobacterium tumefaciens by the electroporation method. Agrobacterium transformant was cultivated at 28° C. until OD value reached 1.0 at 600 nm, centrifuged at 25° C. for 10 minutes at 5,000 rpm, and then cells were harvested. The resulting cells were suspended by using infiltration medium (IM; 1× MS salts, 1× B5 vitamin, 5% sucrose, 0.005% Silwet L-77, Lehle Seed, USA) until final OD value reached 2.0 at 600 nm. 4-weeked Arabidopsis thaliana was submerged to Agrobacterium tumefaciens suspension in a vacuum chamber, left for 10 minutes under 10⁴ Pa of vacuum, and then put in a polyethylene bag for 24 hours. After that, Arabidopsis tumefaciens transformant was cultivated continuously to harvest seed T1. At this moment, wild type Arabidopsis thaliana and Arabidopsis thaliana transformant transformed with the cloning vector pSEN without AtPDX4 gene were adopted as control groups.

<3-2> Characterization of Arabidopsis thaliana Transformant T1 and T2

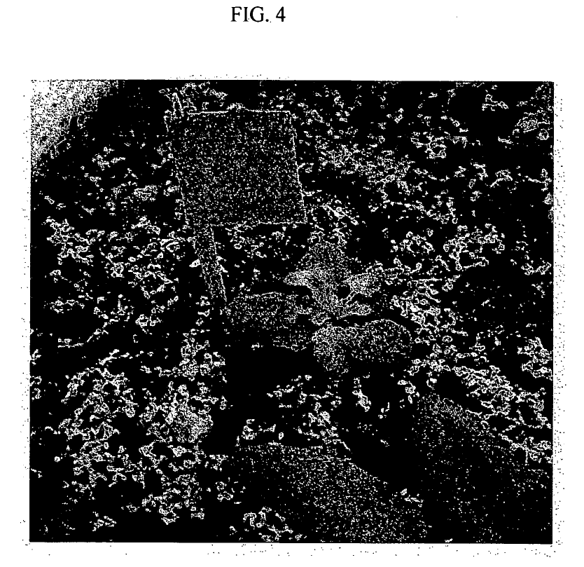

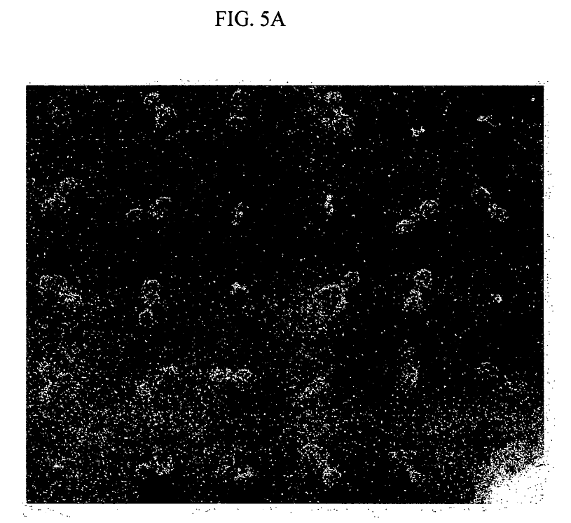

The resulting seed harvested from Arabidopsis thaliana transformant as described in Example 3-1 was submerged in herbicide solution, Basta (Kyungnong Co. Ltd., Korea) for 30 minutes, cultivated and selected. Then, the herbicide Basta was added to pots more 5 times for Arabidopsis thaliana transformant during the growth period to observe the growth pattern of Arabidopsis thaliana. As a result, it is clarified that Arabidopsis thaliana transformant was suppressed severely to grow, contrast to the control group (Arabidopsis transformed with the cloning vector pSEN without anti-sense AtPDX4 gene) and gave rise to leaf yellows from main vein toward edge (See FIG. 4). Also, the anti-sense efficacy against the gene of the present invention was so strong to bring about the mortality of transformant as well as severe growth inhibition and yellow phenomenon.

Meanwhile, in order to investigate whether the plant transformant becomes pyridoxine auxotroph mutant or not after transformed with anti-sense construct against AtPDX4 gene, T2 seed of Arabidopsis thaliana transformant was collected from T1 transformant and cultivated to estimate the reaction by adding pyridoxine. Above all, MS medium containing 12 mg/L PPT (phosphinothricine, Duchefa, Netherlands) was prepared and T2 seeds were treated at a low temperature (4° C.) for 3 days. 120 of T2 seeds were cultivated respectively on 2 (total 4) petri dishes of MS media (30 seeds per petri dish) with or without 2.5 mg/L of pyridoxine-HCl (Sigma, USA), so as to select T2 Arabidopsis thaliana transformant.

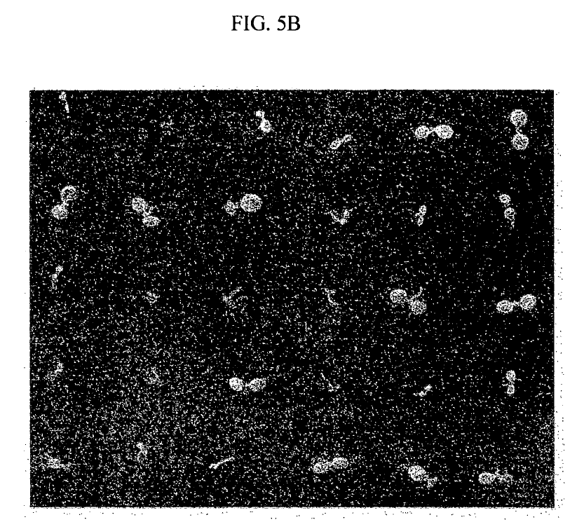

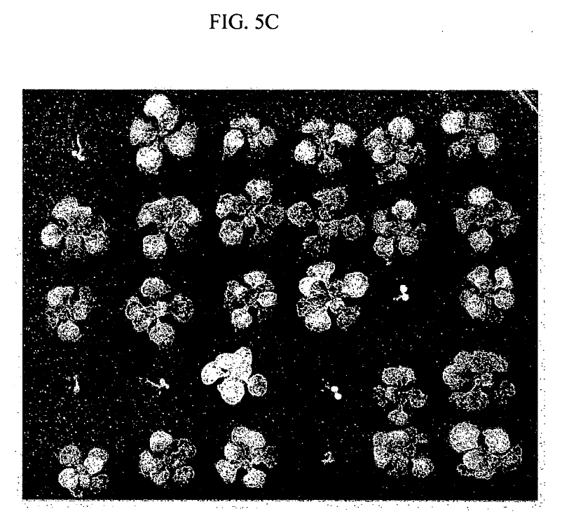

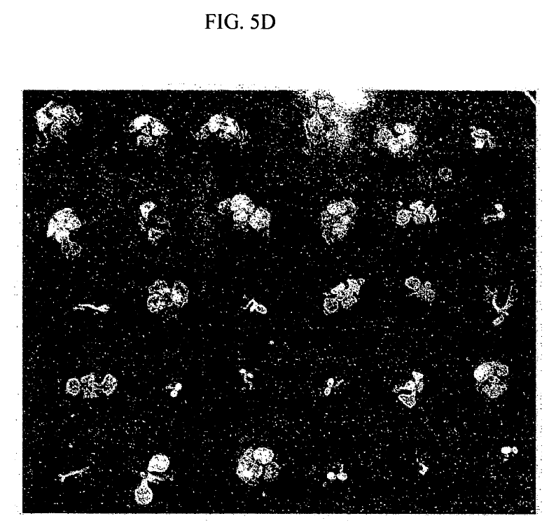

As a result, 22 seeds were germinated to grow when cultivated on petri dishes containing pyridoxine for 7 days and observed not to change in the phenotype remarkably, compared with wild type Arabidopsis thaliana. To the contrary, only 17 seeds were measured to grow when cultivated on petri dishes without pyridoxine. In detail, these were mostly retarded in the growth, seriously caused yellows in whole leaves and became higher in the lethality in more 5 seeds than when cultivated on petri dishes containing pyridoxine (See FIG. 5a and FIG. 5b). Besides, 24 seeds were germinated to grow when cultivated on petri dishes containing pyridoxine for 19 days and estimated not to change in the phenotype such as growth retardation, compared with wild type Arabidopsis, although troubled by yellows slightly. The partial yellows of plant transformant may be caused by pyridoxine content lacking in culture medium. To the contrary, only 19 seeds were observed to grow when cultivated on petri dishes without pyridoxine. In detail, these were mostly retarded in the growth, seriously caused yellows in whole leaves and became higher in the lethality in more 5 seeds than when cultivated on petri dishes containing pyridoxine (See FIG. 5c and FIG. 5d). Especially, the lethal plants may occur according to the normal ratio (mutant: wild type=3:1) in 1 copy of anti-sense line, when cultivated on petri dishes containing pyridoxine. Consequently, the T2 Arabidopsis thaliana transformant has features in the phenotypic aspects to retard seriously in the growth, to cause yellows in whole leaves and to become lethal, and then to recover after treating pyridoxine. Therefore, it is verified that the plant of the present invention transformed with an anti-sense construct against AtPDX4 gene should be a pyridoxine auxotroph mutant.

Furthermore, in order to investigate whether the plant transformant transformed with anti-sense construct against AtPDX4 gene may recover the phenotype by any other vitamin B6's or not, Arabidopsis thaliana transformant was observed in the growth aspects to estimate the reaction by treating pyridoxamine, pyridoxal and/or pyridoxal-5-phosphate in addition to pyridoxine. Above all, in order to select T2 Arabidopsis transformant, MS medium containing 12 mg/L PPT (phosphinothricine, Duchefa, Netherlands) was prepared and T2 seeds were treated at a low temperature (4° C.) for 3 days. 120 of T2 seeds were cultivated on petri dishes containing MS media with or without 0.5 mg/L of pyridoxine-HCl (Sigma, USA), pyridoxamine-2HCl (Sigma, USA), pyridoxal-HCl (Sigma, USA) or pyridoxal-5-phosphate (Sigma, USA).

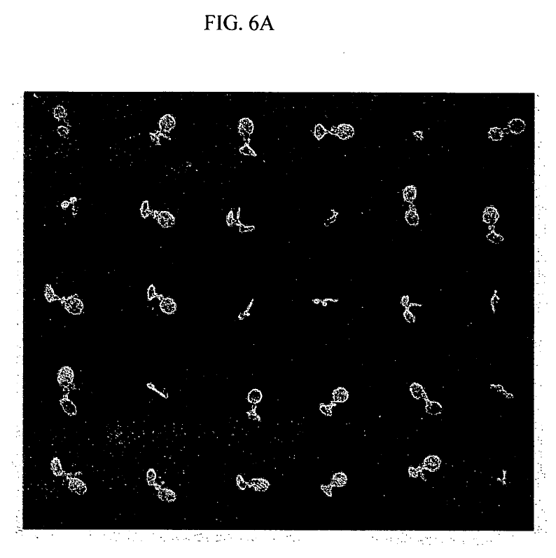

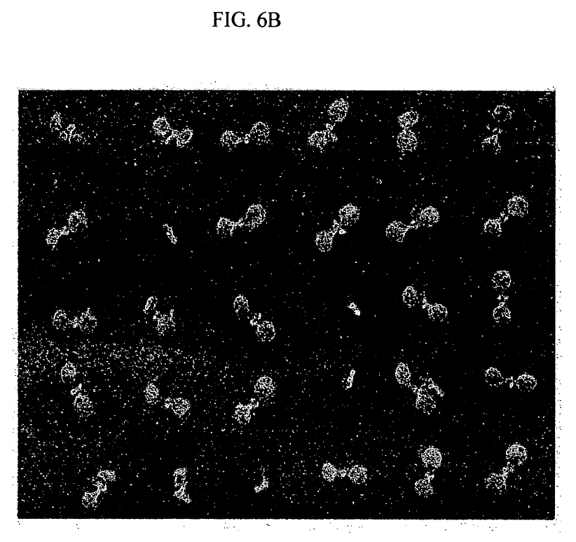

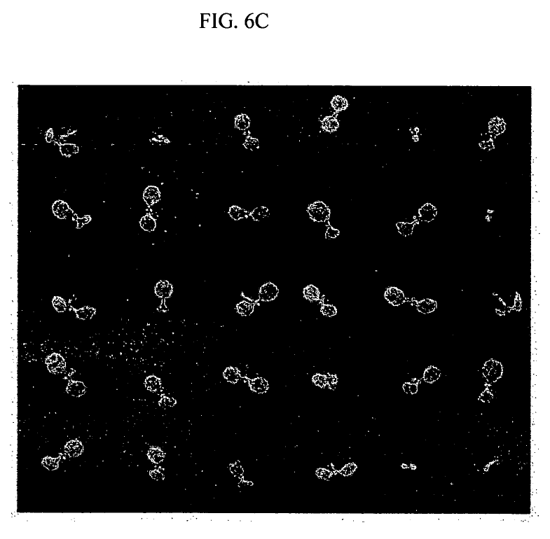

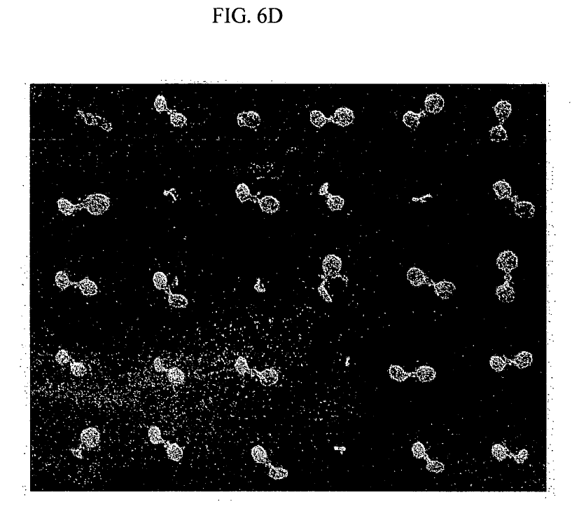

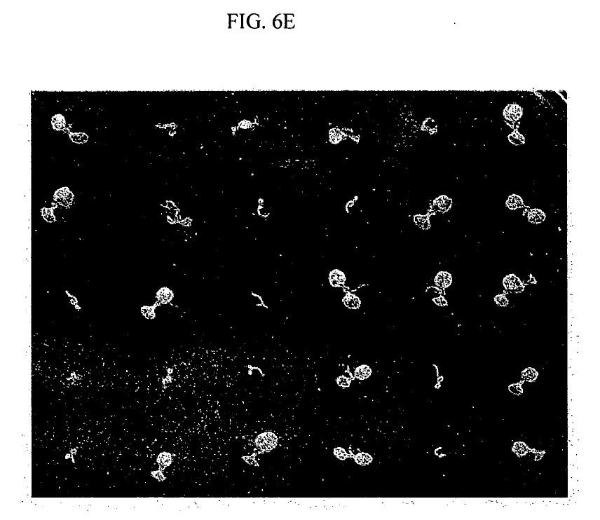

As a result, 26 seeds were germinated to grow when cultivated on petri dishes containing pyridoxine for 7 days and observed not to change in the phenotype remarkably, compared with wild type Arabidopsis. To the contrary, only 21 seeds were measured to grow when cultivated on petri dishes without pyridoxine, mostly retarded in the growth and seriously caused yellows in whole leaves. Besides, it is observed that the plant transformant cultivated in petri dishes containing any other vitamin B6's be similar in the phenotypic aspects to those cultivated on petri dishes without pyridoxine, although recovered from lethal traits in some extent (See FIG. 6a, FIG. 6b, FIG. 6c, FIG. 6d and FIG. 6e). Consequently, it is confirmed that AtPDX4 gene of the present invention may regulate directly the biosynthesis of pyridoxine excluding any other vitamin B6's. Therefore, the polynucleotide of the present invention will be useful for a target gene to develop a novel herbicide.

What is claimed is:

1. A polypeptide participating in pyridoxine biosynthesis, selected from a group comprising (a), (b) and (c) polypeptide:
(a) polypeptide containing all portion of the amino acid sequence set forth in SEQ ID NO. 2;
(b) polypeptide containing a substantial portion of the amino acid sequence set forth in SEQ ID NO. 2;
(c) polypeptide substantially similar to the above (a) or (b) polypeptide

2. A polynucleotide encoding the polypeptide of claim 1.

3. A method for inhibiting a plant growth, which comprises a step of inhibiting the expression or function of a polypeptide that participates in pyridoxine biosynthesis and consists of the amino acid sequence of SEQ ID NO. 2 or its equivalent sequence.

4. The method according to claim 3, in which the step is performed by introducing an anti-sense nucleotide against the polynucleotide set forth in claim 2 into a plant.

5. The method according to claim 3, in which the step is performed by introducing a recombinant vector containing an anti-sense nucleotide against the polynucleotide set forth in claim 2 into a plant.

6. The method according to claim 3, in which the step is performed by introducing Agrobacterium tumefaciens transformant transformed with a recombinant vector containing an anti-sense nucleotide against the polynucleotide set forth in claim 2, into a plant.

7. The method according to claim 3, in which the step is performed by any one technique that is selected among gene deletion, gene insertion, T-DNA insertion, homologous recombination, transposon tagging, small interfering RNA (siRNA) and the like.

8. A process for screening a growth inhibitor of plants, which comprises a step for screening a substance inhibiting the expression or function of polypeptide that participates in pyridoxine biosynthesis and consists of the amino acid sequence of SEQ ID NO. 2 or its equivalent sequence.

9. A composition for inhibiting plant growth comprising a growth inhibitor screened by the process set forth in claim 8.

10. The composition according to claim 9, in which the inhibitor is selected from a group consisting of (1) the anti-sense nucleotide against the polynucleotide of claim 2; (2) the recombinant vector containing the anti-sense nucleotide against the polynucleotide of claim 2; and (3) the transformant of Agrobacterium tumefaciens transformed with the recombinant vector containing the anti-sense nucleotide against the polynucleotide of claim 2.
